SEQUENCE LISTING (14 sequences)

SEQ ID NO: 1 - protein, 404 aa, synthetic construct
  source 1..404: note = Rsp_metZ_long
MGIAFREGRTGMTKDWKTRTQLVHGGSRRSQYGEMAEAIFLTQGFVYDSAEQAEARFIETGADEFIYARYGNPTTRMFEERIAAVEGTEDAFATASGMAAIHGVLTSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKVLEPFMKHTGGSMSPFNAWLMLNGMATLDLRCRAMADTAEKIARALEGHPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIADLKQALAVI

SEQ ID NO: 2 - protein, 404 aa, synthetic construct
  source 1..404: note = Rsp_metZ_long_I3N+F65Y+V104A
MGNAFREGRTGMTKDWKTRTQLVHGGSRRSQYGEMAEAIFLTQGFVYDSAEQAEARFIETGADEYIYARYGNPTTRMFEERIAAVEGTEDAFATASGMAAIHGALTSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKVLEPFMKHTGGSMSPFNAWLMLNGMATLDLRCRAMADTAEKIARALEGHPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIADLKQALAVI

SEQ ID NO: 3 - protein, 404 aa, synthetic construct
  source 1..404: note = Rsp_metZ_long_I3N
MGNAFREGRTGMTKDWKTRTQLVHGGSRRSQYGEMAEAIFLTQGFVYDSAEQAEARFIETGADEFIYARYGNPTTRMFEERIAAVEGTEDAFATASGMAAIHGVLTSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKVLEPFMKHTGGSMSPFNAWLMLNGMATLDLRCRAMADTAEKIARALEGHPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIADLKQALAVI

SEQ ID NO: 4 - protein, 404 aa, synthetic construct
  source 1..404: note = Rsp_metZ_long_F65Y
MGIAFREGRTGMTKDWKTRTQLVHGGSRRSQYGEMAEAIFLTQGFVYDSAEQAEARFIETGADEYIYARYGNPTTRMFEERIAAVEGTEDAFATASGMAAIHGVLTSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKVLEPFMKHTGGSMSPFNAWLMLNGMATLDLRCRAMADTAEKIARALEGHPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIADLKQALAVI

SEQ ID NO: 5 - protein, 404 aa, synthetic construct
  source 1..404: note = Rsp_metZ_long_V104A
MGIAFREGRTGMTKDWKTRTQLVHGGSRRSQYGEMAEAIFLTQGFVYDSAEQAEARFIETGADEFIYARYGNPTTRMFEERIAAVEGTEDAFATASGMAAIHGALTSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKVLEPFMKHTGGSMSPFNAWLMLNGMATLDLRCRAMADTAEKIARALEGHPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIADLKQALAVI

SEQ ID NO: 6 - protein, 393 aa, synthetic construct
  source 1..393: note = Rsp_metZ_WT
MTKDWKTRTQLVHGGSRRSQYGEMAEAIFLTQGFVYDSAEQAEARFIETGADEFIYARYGNPTTRMFEERIAAVEGTEDAFATASGMAAIHGVLTSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKVLEPFMKHTGGSMSPFNAWLMLNGMATLDLRCRAMADTAEKIARALEGHPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIADLKQALAVI

SEQ ID NO: 7 - protein, 404 aa, synthetic construct
  source 1..404: note = Rsp_metZ_long_V196T
MGIAFREGRTGMTKDWKTRTQLVHGGSRRSQYGEMAEAIFLTQGFVYDSAEQAEARFIETGADEFIYARYGNPTTRMFEERIAAVEGTEDAFATASGMAAIHGVLTSIVRAGDHLVAARALFGSCIYILEEVLGRFGVEVTFVDGTDLDQWRAAVRPGTKAVFFESVSNPTLEVADIGAIAEIAHAVGALVIVDNTFATPVFSTAVRQGADVVIYSATKHIDGQGRALGGVVCASQAFIRKVLEPFMKHTGGSMSPFNAWLMLNGMATLDLRCRAMADTAEKIARALEGHPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIADLKQALAVI

SEQ ID NO: 8 - protein, 404 aa, synthetic construct
  source 1..404: note = Variant
MGXXFREGRTGMTKDWKTRTQLVHGGSRRSQYGEMAEAIFLTQGFVYDSAEQAEARFIETGADEXIYARYGNPTTRMFEERIAAVEGTEXAFATASGMAAIHGXLXSIVRAGDHLVAARALFGSCIYILEEVLGRFGVEVTFVDGTDLDQWRAAVRPGTKAVFFESVSNPTLEVADIGAIAEIAHAVGALVIVDNXFATPVFSTAVRQGADVVIYSATKHIDGQGRALGGVVCASQAFIRKXLEPFMKHTGGSMSPFNAWXMLNGMATLDLRCRAMADTAEKIARALEGXPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIXDLKQALAVI

SEQ ID NO: 9 - DNA, 1215 bp, synthetic construct
  source 1..1215: mol_type = other DNA; note = Rsp_metZ_long nt
atgggtatcgcgtttcgtgaaggacggacgggcatgacgaaggactggaagacaaggacgcaactcgtccacgggggcagccgccggagccagtatggcgaaatggccgaggcgatcttcctgacccagggcttcgtctacgactcggccgaacaggccgaagcgcgcttcatcgagaccggcgccgacgaattcatctatgcccgctacggcaaccccacgacgcgcatgttcgaagagcgcatcgcggccgtcgagggcaccgaggatgcgttcgccaccgcctcgggcatggccgcgatccacggcgtgctcacctcgatcgtgcgggcgggcgatcatctggtggcggcgcgcgctctgttcggctcctgcatctacatcctcgaggaggtgctgggccgattcggcgtcgaggtgaccttcgtcgacggcaccgatctcgatcagtggcgcgcggcggtgcggcccggcacgaaggccgtgttcttcgagtcggtctcgaatccgacgctcgaggtggccgatatcggcgccatcgccgagatcgcccatgccgtgggcgcgctcgtcatcgtggacaatgtcttcgcgacgcccgtcttctcgacggcggtgcggcagggcgcggatgtggtgatctattcggccaccaagcacatcgacgggcaagggcgcgcgctcggcggcgtggtctgcgcctcgcaggccttcatccgcaaggtgctcgaacccttcatgaagcacaccggcggctcgatgagccccttcaacgcctggctcatgctgaacgggatggcgacgctcgacctgcgctgccgcgcgatggccgacacggccgagaagatcgcccgcgcgctcgagggccatccgcagctcggccgcgtgatccatcccgcgctggaaagccacccgcagcacgagatggccaaggcgcagatggagcgtcccggcacgatgatcgcgctcgacctcgccgggggcaaggaggcggccttccgcttcctcgacgccctgaggatcgtgaagatctccaacaatctgggcgatgcccgctcgatcgcgacccacccggcaacgaccacccaccagcgtctttccgacgcgcagaaggcccatctcggcatcacgcccgggctcgtgcggctgtcggtggggctcgaggatgcggacgacctgatcgccgatctgaaacaggcgctcgcggtgatctga

SEQ ID NO: 10 - DNA, 1215 bp, synthetic construct
  source 1..1215: mol_type = other DNA; note = Rsp_metZ_long_I3N+F65Y+V104A nt
atgggtaacgcgtttcgtgaaggacggacgggcatgacgaaggactggaagacaaggacgcaactcgtccacgggggcagccgccggagccagtatggcgaaatggccgaggcgatcttcctgacccagggcttcgtctacgactcggccgaacaggccgaagcgcgcttcatcgagaccggcgccgacgaatacatctatgcccgctacggcaaccccacgacgcgcatgttcgaagagcgcatcgcggccgtcgagggcaccgaggatgcgttcgccaccgcctcgggcatggccgcgatccacggcgcgctcacctcgatcgtgcgggcgggcgatcatctggtggcggcgcgcgctctgttcggctcctgcatctacatcctcgaggaggtgctgggccgattcggcgtcgaggtgaccttcgtcgacggcaccgatctcgatcagtggcgcgcggcggtgcggcccggcacgaaggccgtgttcttcgagtcggtctcgaatccgacgctcgaggtggccgatatcggcgccatcgccgagatcgcccatgccgtgggcgcgctcgtcatcgtggacaatgtcttcgcgacgcccgtcttctcgacggcggtgcggcagggcgcggatgtggtgatctattcggccaccaagcacatcgacgggcaagggcgcgcgctcggcggcgtggtctgcgcctcgcaggccttcatccgcaaggtgctcgaacccttcatgaagcacaccggcggctcgatgagccccttcaacgcctggctcatgctgaacgggatggcgacgctcgacctgcgctgccgcgcgatggccgacacggccgagaagatcgcccgcgcgctcgagggccatccgcagctcggccgcgtgatccatcccgcgctggaaagccacccgcagcacgagatggccaaggcgcagatggagcgtcccggcacgatgatcgcgctcgacctcgccgggggcaaggaggcggccttccgcttcctcgacgccctgaggatcgtgaagatctccaacaatctgggcgatgcccgctcgatcgcgacccacccggcaacgaccacccaccagcgtctttccgacgcgcagaaggcccatctcggcatcacgcccgggctcgtgcggctgtcggtggggctcgaggatgcggacgacctgatcgccgatctgaaacaggcgctcgcggtgatctga

SEQ ID NO: 11 - DNA, 3728 bp, synthetic construct
  source 1..3728: mol_type = other DNA; note = pUCtk
gatctctccttcacagattcccaatctcttgttaaataacgaaaaagcatcaatcaaaacggcggcatgtctttctatattccagcaatgttttataggggacatattgatgaagatgggtatcaccttagtaaaaaaagaattgctataagctgctcttttttgttcgtgatatactgataataaattgaattttcacacttctggaaaaaggagatataccgatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagctt

SEQ ID NO: 12 - protein, 404 aa, synthetic construct
  source 1..404: note = D90A
MGNAFREGRTGMTKDWKTRTQLVHGGSRRSQYGEMAEAIFLTQGFVYDSAEQAEARFIETGADEYIYARYGNPTTRMFEERIAAVEGTEAAFATASGMAAIHGALTSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKVLEPFMKHTGGSMSPFNAWLMLNGMATLDLRCRAMADTAEKIARALEGHPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIADLKQALAVI

SEQ ID NO: 13 - protein, 404 aa, synthetic construct
  source 1..404: note = QM
MGNKFREGRTGMTKDWKTRTQLVHGGSRRSQYGEMAEAIFLTQGFVYDSAEQAEARFIETGADEYIYARYGNPTTRMFEERIAAVEGTEAAFATASGMAAIHGALCSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKVLEPFMKHTGGSMSPFNAWLMLNGMATLDLRCRAMADTAEKIARALEGCPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIADLKQALAVI

SEQ ID NO: 14 - protein, 404 aa, synthetic construct
  source 1..404: note = Variant 3
MGNKFREGRTGMTKDWKTRTQLVHGGSRRSQYGEMAEAIFLTQGFVYDSAEQAEARFIETGADEYIYARYGNPTTRMFEERIAAVEGTEAAFATASGMAAIHGALCSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKLLEPFMKHTGGSMSPFNAWVMLNGMATLDLRCRAMADTAEKIARALEGCPQLGRVIHPALESHPQHEMAKAQMERPGTMIALDLAGGKEAAFRFLDALRIVKISNNLGDARSIATHPATTTHQRLSDAQKAHLGITPGLVRLSVGLEDADDLIVDLKQALAVI

(19) United States
(12) Patent Application Publication
(10) Pub. No.: US 2026/0242755 A1
(43) Pub. Date: Aug. 20, 2026

(54) O-ACYLHOMOSERINE SULFHYDRYLASE VARIANT AND USE THEREOF

(71) Applicant: CJ CHEILJEDANG CORPORATION, Seoul (KR)

(72) Inventors: Bo Ram LIM, Seoul (KR); Minsun HAN, Seoul (KR); Joon Young JUNG, Seoul (KR); Bo Seong PARK, Seoul (KR); Yong Uk SHIN, Seoul (KR); Min Ji BAEK, Seoul (KR); So Young KIM, Seoul (KR); Vivian YU, Emeryville, CA (US); Doug HATTENDORF, Emeryville, CA (US); Stacy-Anne MORGAN, Emeryville, CA (US)

(73) Assignee: CJ CHEILJEDANG CORPORATION, Seoul (KR)

(21) Appl. No.: 19/152,419

(22) PCT Filed: Jan. 31, 2024
(86) PCT No.: PCT/KR2024/001443

§ 371 Date: Jul. 30, 2025

Related U.S. Application Data
(60) Provisional application No. 63/442,197, filed on Jan. 31, 2023.

(51) Int. Cl. C12N 9/10; C12P 13/12

(52) U.S. Cl. CPC C12N 9/1085; C12P 13/12; C12Y 205/01049

(57) ABSTRACT

Provided are an O-acylhomoserine sulfhydrylase variant and use thereof.

### TECHNICAL FIELD

The present disclosure relates to an O-acylhomoserine sulfhydrylase variant and use thereof.

### BACKGROUND ART

Methionine, which is one of the essential amino acids for use in protein synthesis, is widely used as feed and food additives, and also used as a food material, a nutrient for plants, and a material for pharmaceuticals. Methionine plays an important role in transmethylation in vivo, and is converted to cysteine via homocysteine and cystathionine. Further, since methionine serves as a sulfur donor, it plays an important role in the enzymatic conversion process involved in transsulfuration.

Homocysteine is an intermediate metabolite generated during methionine metabolism in vivo, and a kind of amino acids not forming proteins. Homocysteine is generally converted to cysteine or methionine in vivo, and exists only in small amounts. Since homocysteine functions as a strong oxidizing agent, it may cause problems such as cytotoxicity when excessively accumulated in the body, but may be widely used in feed or food additives, food materials, plant nutrients, biopolymers, pharmaceutical fields, etc.

A technique that allows production of methionine and homocysteine using a biological method is disclosed in WO2008/013432. This method, named as a two-step method, includes a process of producing a precursor by fermentation and a subsequent process of converting the precursor by enzymes.

Development of the two-step method resolves all of the existing problems such as toxicity of a substrate specific to sulfide, feedback control in a strain by methionine and SAM, and decomposition of intermediate products specific to cystathionine gamma synthase, O-succinylhomoserine sulfhydrylase, and O-acetylhomoserine sulfhydrylase. Furthermore, this method, which enables selective production of only L-methionine, is superior to the existing chemical synthesis process that produces both of D- and L-methionine at the same time, and this method enables additional production of organic acids, more specifically, succinic acid and acetic acid at the same time, as by-products through the same reaction.

The two-step enzymatic conversion process employs enzymes having the activity of cystathionine gamma synthase, O-succinylhomoserine sulfhydrylase, or O-acetylhomoserine sulfhydrylase. In the two-step enzymatic conversion process, enzymes capable of efficiently producing products are needed.

### DISCLOSURE

### Technical Solution

An object of the present disclosure is to provide an O-acylhomoserine sulfhydrylase variant, in which one or more of amino acids corresponding to positions 4, 90, 106, 242, 261, 290, and 395 from the N-terminus of SEQ ID NO: 1 are each substituted with another amino acid.

Another object of the present disclosure is to provide a polynucleotide encoding the variant.

Still another object of the present disclosure is to provide a microorganism including an O-acylhomoserine sulfhydrylase variant, in which one or more of amino acids corresponding to positions 4, 90, 106, 242, 261, 290, and 395 from the N-terminus of SEQ ID NO: 1 are each substituted with another amino acid, or a polynucleotide encoding the same.

Still another object of the present disclosure is to provide a method of producing L-methionine or a precursor thereof, the method including the step of bringing O-acylhomoserine into contact with an O-acylhomoserine sulfhydrylase variant, in which one or more of amino acids corresponding to positions 4, 90, 106, 242, 261, 290, and 395 from the N-terminus of SEQ ID NO: 1 are each substituted with another amino acid; or a microorganism including the variant or a polynucleotide encoding the same.

### Advantageous Effects

A novel variant of the present disclosure may be used to produce L-methionine and/or a precursor thereof in a high yield.

### DESCRIPTION OF DRAWINGS

FIG. 1 shows a graph showing activities of enzyme variants utilizing sodium methyl mercaptan as a substrate;

FIG. 2 shows activities of 40 kinds of enzyme variants showing excellent titers, which were obtained through screening, as compared to those of controls (Negative control indicates a negative control without acylhomoserine sulfhydrylase enzyme, and Standard control indicates acylhomoserine sulfhydrylase of SEQ ID NO: 2);

FIG. 3 shows methionine production over time according to activities of variant enzymes (D90A, QM);

FIG. 4 shows results of methionine production reaction of variant enzymes (D90A, QM) over pre-treatment time under different pH conditions;

FIG. 5 shows results of methionine production reaction of variant enzyme (Variant 3) over pre-treatment time under different pH conditions;

FIG. 6 shows SDS-PAGE showing expression of control enzyme and variant enzymes (QM and Variant 3); and

FIG. 7 shows a graph showing methionine conversion activities of control enzyme and Variant 3.

### BEST MODE

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below. Further, a number of papers and patent documents are referenced and cited throughout this specification. The disclosures of the cited papers and patent documents are incorporated herein by reference in their entirety to further clarify the level and scope of the subject matter to which the present disclosure pertains.

Further, those skilled in the art will recognize or be able to ascertain using no more than routine experimentation, many equivalents to the specific embodiments of the disclosure described herein. Further, these equivalents should be interpreted to fall within the present disclosure.

One aspect of the present disclosure provides an O-acylhomoserine sulfhydrylase variant, in which one or more of amino acids corresponding to positions 4, 90, 106, 242, 261, 290, and 395 from the N-terminus of SEQ ID NO: 1 are each substituted with another amino acid.

As used herein, “acylhomoserine” refers to a compound in which an acyl group is bound to homoserine, and includes both succinylhomoserine and acetylhomoserine. For example, the acylhomoserine may be O-succinylhomoserine or O-acetylhomoserine.

In the present disclosure, O-acylhomoserine sulfhydrylase may be a protein having activity of one or more of O-acetylhomoserine sulfhydrylase, O-acetylhomoserine transsulfurase, O-succinylhomoserine sulfhydrylase, and O-succinylhomoserine transsulfurase.

As used herein, the term “sulfhydrylation” is used interchangeably with “sulfhydration” and refers to a reaction that provides a sulfhydryl (—SH) functional group to a specific molecule. This term may refer to a reaction in the methionine synthesis process with respect to the objects of the present disclosure, but is not limited thereto. An enzyme involved in the “sulfhydration” may be referred to as “sulfhydrylase”.

As used herein, the term “O-acetylhomoserine” is the first specific intermediate in biosynthesis of methionine, which is found in microorganisms, and may be generated from L-homoserine and acetyl-CoA by homoserine acetyltransferase catalysis at the bifurcation with biosynthesis of threonine.

In the present disclosure, “O-acetylhomoserine sulfhydrylase” refers to an enzyme capable of synthesizing L-methionine using O-acetylhomoserine, which is a precursor of L-methionine, and methyl mercaptan. The O-acetylhomoserine sulfhydrylase may have the following three activities.

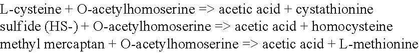

As used herein, “O-succinylhomoserine sulfhydrylase” refers to an enzyme capable of synthesizing L-methionine using O-succinylhomoserine and methyl mercaptan. The O-succinylhomoserine sulfhydrylase may have the following three activities.

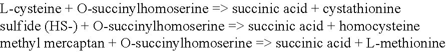

Some enzymes named O-acetylhomoserine sulfhydrylase may also utilize O-succinylhomoserine as a substrate, and also have O-succinylhomoserine sulfhydrylase activity. In one embodiment, the O-acylhomoserine sulfhydrylase of the present disclosure may also have O-acetylhomoserine sulfhydrylase and O-succinylhomoserine sulfhydrylase activities, but is not necessarily limited thereto.

The O-acylhomoserine sulfhydrylase of the present disclosure may be O-acylhomoserine sulfhydrylase which may be modified to prepare the O-acylhomoserine sulfhydrylase provided in the present disclosure or a polypeptide having the activity of O-acylhomoserine sulfhydrylase.

Specifically, the O-acylhomoserine sulfhydrylase may be a naturally occurring polypeptide or wild-type polypeptide, may be a mature polypeptide thereof, and may include a variant or functional fragment thereof. It may include any one without limitation, as long as it may be a parent of the O-acylhomoserine sulfhydrylase variant of the present disclosure.

In one embodiment, the O-acylhomoserine sulfhydrylase of the present disclosure may be derived from a microorganism of the family Rhodobacteraceae. In one embodiment, the O-acylhomoserine sulfhydrylase may be derived from a microorganism of the Rhodobacter sp. In another embodiment, the O-acylhomoserine sulfhydrylase may be derived from a microorganism of the Cereibacter sp. In one embodiment of the above-described embodiments, the O-acylhomoserine sulfhydrylase of the present disclosure may be derived from Rhodobacter sphaeroides. Meanwhile, Rhodobacter sphaeroides may also be named Luteovulum sphaeroides or Cereibacter sphaeroides, and the name of the microorganism is not limited to those described above, and may include any one without limitation, as long as it can be classified into the same taxon as Rhodobacter sphaeroides.

In one embodiment, in the O-acylhomoserine sulfhydrylase to be modified in the present disclosure, the amino acids corresponding to positions 4, 90, 106, 242, 261, 290, and 395 from the N-terminus of SEQ ID NO: 1 may be alanine (A), aspartic acid (D), threonine (T), valine (V), leucine (L), histidine (H), and/or alanine (A), respectively.

In one embodiment, the O-acylhomoserine sulfhydrylase of the present disclosure may be the polypeptide of SEQ ID NO: 1. In one embodiment of the above-described embodiments, the O-acylhomoserine sulfhydrylase of the present disclosure may be a polypeptide having about 60%, 70%, 75%, 80%, 85%, 90%, 93%, 95%, 96%, 97%, 98%, or 99% or more sequence identity to the polypeptide of SEQ ID NO: 1, but is not limited thereto. As long as the polypeptide has activity equal or corresponding to the polypeptide consisting of the amino acid sequence of SEQ ID NO: 1, it is included in the scope of O-acylhomoserine sulfhydrylase. It is also apparent that any polypeptide/protein having an amino acid sequence, in which part of the sequence is deleted, modified, substituted, or added, may also be included within the scope of the polypeptide/protein of the present disclosure to be modified, as long as it has such a homology or identity and exhibits the O-acylhomoserine sulfhydrylase activity.

For example, a polypeptide (WP_002722742.1) of SEQ ID NO: 6 is a polypeptide having an amino acid sequence, in which part of the sequence is deleted, as compared to the polypeptide of SEQ ID NO: 1, and exhibiting the O-acylhomoserine sulfhydrylase activity, and is included within the scope of the polypeptide of the present disclosure to be modified. For another example, polypeptides of SEQ ID NO: 2, 3, 4, 5, and 7 are polypeptides having an amino acid sequence, in which part of the sequence is substituted, as compared to the polypeptide of SEQ ID NO: 1, and exhibiting the O-acylhomoserine sulfhydrylase activity, and are included within the scope of the polypeptide of the present disclosure to be modified.

For one embodiment, with regard to the O-acylhomoserine sulfhydrylase to be modified in the present disclosure, any one or more amino acids of the amino acids corresponding to positions 3, 65, 104, and 196 from the N-terminus of SEQ ID NO: 1 may be amino acids selected from glycine, alanine, valine, leucine, isoleucine, methionine, phenylalanine, tryptophan, proline, serine, threonine, cysteine, tyrosine, asparagine, glutamine, aspartic acid, glutamic acid, lysine, arginine, and histidine.

In any one embodiment of the above-described embodiments, with regard to the O-acylhomoserine sulfhydrylase to be modified in the present disclosure, the amino acid corresponding to position 3 from the N-terminus of SEQ ID NO: 1 may be isoleucine or an amino acid other than isoleucine. For example, the amino acid corresponding to position 3 may be asparagine (N).

In any one embodiment of the above-described embodiments, with regard to the O-acylhomoserine sulfhydrylase to be modified in the present disclosure, the amino acid corresponding to position 65 from the N-terminus of SEQ ID NO: 1 may be phenylalanine or an amino acid other than phenylalanine. For example, the amino acid corresponding to position 65 may be tyrosine (Y).

In any one embodiment of the above-described embodiments, with regard to the O-acylhomoserine sulfhydrylase to be modified in the present disclosure, the amino acid corresponding to position 104 from the N-terminus of SEQ ID NO: 1 may be valine (V) or an amino acid other than valine. For example, the amino acid corresponding to position 104 may be alanine (A).

In any one embodiment of the above-described embodiments, with regard to the O-acylhomoserine sulfhydrylase to be modified in the present disclosure, the amino acid corresponding to position 196 from the N-terminus of SEQ ID NO: 1 may be valine or an amino acid other than valine. For example, the amino acid corresponding to position 196 may be threonine (T).

In any one embodiment of the above-described embodiments, with regard to the O-acylhomoserine sulfhydrylase to be modified in the present disclosure, the amino acids corresponding to positions 3, 65, and 104 from the N-terminus of SEQ ID NO: 1 may be asparagine (N), tyrosine (Y), and alanine (A), respectively.

In any one embodiment of the above-described embodiments, the O-acylhomoserine sulfhydrylase to be modified in the present disclosure may have an amino acid sequence represented by SEQ ID NO: 1, 2, 3, 4, 5, 6, or 7, or may include the same, or may consist of the amino acid sequence or may essentially consist of the amino acid sequence. In any one embodiment of the above-described embodiments, the O-acylhomoserine sulfhydrylase may include an amino acid sequence having about 60% or higher, for example, about 60%, 70%, 75%, 80%, 85%, 90%, 93%, 95%, 96%, 97%, 98%, 98.5%, or 99% or higher sequence homology or identity to the polypeptide of SEQ ID NO: 1, 2, 3, 4, 5, 6, or 7, but is not limited thereto.

As used herein, the term “homology” or “identity” refers to a degree of relevance between two given amino acid sequences or nucleotide sequences, and it may be expressed as a percentage. The terms homology and identity may often be used interchangeably.

A sequence homology or identity of conserved polynucleotides or polypeptides may be determined by standard alignment algorithm, and default gap penalties established by a program being used may be used together. Actually, homologous or identical sequences may hybridize with each other along the entire or part of the sequence under moderate or highly stringent conditions. It is obvious that hybridization also includes hybridization with a polynucleotide containing general codons or codons considering codon degeneracy.

Whether any two polynucleotide- or polypeptide sequences have a homology, similarity, or identity may be determined using computer algorithms known in the art, e.g., “FASTA” program using default parameters as in Pearson et al. (1988) [Proc. Natl. Acad. Sci. USA 85]: 2444. Alternatively, Needleman-Wunsch algorithm (Needleman and Wunsch, 1970, J. Mol. Biol. 48:443-453) as performed in a Needleman program of EMBOSS package (EMBOSS: The European Molecular Biology Open Software Suite, Rice et al., 2000, Trends Genet. 16:276-277) (version 5.0.0 or a later version) may be used to determine the same (including GCG program package (Devereux, J., et al, Nucleic Acids Research 12:387 (1984)), BLASTP, BLASTN, FASTA (Atschul, [S.] [F.,] [et al., J Mol Biol 215]: 403 (1990); Guide to Huge Computers, Martin J. Bishop, [ED.,] Academic Press, San Diego, 1994, and [CARILLO et al.] (1988) SIAM J Applied Math 48:1073). For example, the homology, similarity, or identity may be determined using BLAST from the National Center for Biotechnology Information database or ClustalW.

The homology, similarity, or identity between polynucleotides or polypeptides may be determined, for example, by comparing the sequence information using a GAP computer program, such as a program as in Needleman et al. (1970), J Mol Biol. 48:443, as disclosed by Smith and Waterman, Adv. Appl. Math (1981) 2:482. In brief, the GAP program defines a homology, similarity, or identity as the number of similar aligned symbols (i.e., nucleotides or amino acids) divided by the total number of the symbols in a shorter of the two sequences. The default parameters for the GAP program may include: (1) a binary comparison matrix (including a value 1 for identity and a value 0 for non-identity) and the weighted comparison matrix of Gribskov et al. (1986) Nucl. Acids Res. 14:6745, as described in Schwartz and Dayhoff, eds., Atlas Of Protein Sequence And Structure, National Biomedical Research Foundation, pp. 353-358 (1979) or EDNAFULL (EMBOSS version of NCBI NUC4.4) substitution matrix); (2) a penalty of 3.0 for each gap and an additional 0.10 penalty for each symbol in each gap (or a gap open penalty of 10 and a gap extension penalty of 0.5); and (3) no penalty for end gaps.

As used herein, the term “variant” refers to a polypeptide, in which one or more amino acids in the conservative substitution and/or modification is different from the amino acid sequence of the variant before modification, but the functions or properties are maintained. Such a variant may be generally identified by modifying one or more amino acids in the amino acid sequence of the polypeptide, and by evaluating the properties of the modified polypeptide. In other words, ability of the variant may be increased, unchanged, or reduced, as compared to that of the polypeptide before modification. Further, some variants may include variants in which one or more parts such as an N-terminal leader sequence or a transmembrane domain are removed. Other variants may include variants in which part is removed from the N- and/or C-terminus of the mature protein. The term “variant” may be used interchangeably with a variant, modification, modified polypeptide, modified protein, mutant, mutein, divergent, etc., and is not limited as long as the term refers to modification.

Further, the variants may include deletion or addition of amino acids having minimal impact on properties and secondary structure of a polypeptide. For example, the polypeptide may be conjugated to a signal (or leader) sequence at the N-terminus of the variant, which is involved in the translocation of proteins co-translationally or post-translationally. Further, the variant may also be conjugated to another sequence or a linker to identify, purify, or synthesize the variant.

The O-acylhomoserine sulfhydrylase variant provided in the present disclosure may refer to a polypeptide having an arbitrary sequence and the activity of O-acylhomoserine sulfhydrylase; or a variant including substitution of another amino acid for one or more of the amino acids corresponding to positions 4, 90, 106, 242, 261, 290, and 395 from the N-terminus of SEQ ID NO: 1 in the O-acylhomoserine sulfhydrylase.

The O-acylhomoserine sulfhydrylase variant provided in the present disclosure may be a polypeptide including an amino acid sequence, in which one or more of the amino acids corresponding to positions 4, 90, 106, 242, 261, 290, and 395 from the N-terminus of SEQ ID NO: 1 are each substituted with another amino acid.

The “another amino acid” is not limited, as long as it is different from the amino acid before substitution. Meanwhile, in the present disclosure, when it is expressed that ‘a specific amino acid is substituted’, it is obvious that the amino acid is substituted with an amino acid different from the amino acid before the substitution, even though it is not separately indicated that the amino acid is substituted with another amino acid.

Amino acids may be generally classified, based on similarities in polarity, charge, solubility, hydrophobicity, hydrophilicity, and/or amphipathic nature of residues.

For one example, positively charged (basic) amino acids may include arginine, lysine, and histidine; negatively charged (acidic) amino acids may include glutamic acid and aspartic acid; non-polar amino acids having non-polar side chains may include glycine, alanine, valine, leucine, isoleucine, methionine, phenylalanine, tryptophan, and proline; polar amino acids having polar or hydrophilic side chains may include serine, threonine, cysteine, tyrosine, asparagine, and glutamine. For another example, amino acids having electrically charged side chains (electrically charged amino acids) may include arginine, lysine, histidine, glutamic acid, aspartic acid, and amino acids having uncharged side chains (uncharged amino acid; also called neutral amino acids) may include glycine, alanine, valine, leucine, isoleucine, methionine, phenylalanine, tryptophan, proline, serine, threonine, cysteine, tyrosine, asparagine and glutamine. For still another example, phenylalanine, tryptophan, and tyrosine may be classified into aromatic amino acids. For still another example, valine, leucine, isoleucine may be classified into branched amino acid. For still another example, 20 kinds of amino acids are classified, based on their size, into 5 groups in ascending order of volume: glycine, alanine, serine; cysteine, proline, threonine, aspartic acid, asparagine; valine, histidine, glutamic acid, glutamine; isoleucine, leucine, methionine, lysine, arginine; and phenylalanine, tryptophan, tyrosine, but are not limited thereto.

As used herein, the term “corresponding to” refers to an amino acid residue which is at the position recited in a polypeptide, or an amino acid residue which is similar, identical, or homologous to the residue recited in a polypeptide. Identifying the amino acid at the corresponding position may be determining a specific amino acid in a specific reference sequence. As used herein, the term “corresponding region” generally refers to a similar or corresponding position in a related protein or reference protein.

For example, an arbitrary amino acid sequence is aligned with SEQ ID NO: 1, and based on this, each amino acid residue of the amino acid sequence may be numbered with reference to the position of the amino acid residue corresponding to the amino acid residue of SEQ ID NO: 1. For example, a sequence alignment algorithm, as described in the present disclosure, may determine positions of amino acids, or positions, where modifications such as substitution, insertion or deletion occur, by comparing with a query sequence (also referred to as a “reference sequence”).

Such alignment may be performed using, for example, a Needleman-Wunsch algorithm (Needleman and Wunsch, 1970, J. Mol. Biol. 48:443-453), a Needle program of EMBOSS package (EMBOSS: The European Molecular Biology Open Software Suite, Rice et al., 2000), Trends Genet. 16:276-277), etc., but is not limited thereto. A sequence alignment program, a pairwise sequence comparison algorithm, etc., which is known in the art, may be appropriately used.

In one embodiment, the O-acylhomoserine sulfhydrylase variant provided in the present disclosure may include an amino acid sequence, in which 1, 2, 3, 4, 5, 6, or 7 amino acid(s) corresponding to positions 4, 90, 106, 242, 261, 290, and 395 from the N-terminus of SEQ ID NO: 1 is/are substituted with another amino acid(s).

In any one embodiment of the above-described embodiments, the O-acylhomoserine sulfhydrylase variant provided in the present disclosure may include substitution of the amino acid corresponding to position 90 from the N-terminus of SEQ ID NO: 1 with another amino acid.

In any one embodiment of the above-described embodiments, in the variant, the amino acid corresponding to position 90 from the N-terminus of SEQ ID NO: 1 may be substituted with a nonpolar amino acid. For example, the amino acid may be an amino acid selected from glycine, alanine, valine, leucine, isoleucine, methionine, phenylalanine, tryptophan, and proline. In any one embodiment of the above-described embodiments, the variant may be a variant, in which the amino acid corresponding to position 90 from the N-terminus of SEQ ID NO: 1 is substituted with alanine (A).

In any one embodiment of the above-described embodiments, the O-acylhomoserine sulfhydrylase variant provided in the present disclosure may include substitution of the amino acid corresponding to position 4 from the N-terminus of SEQ ID NO: 1 with another amino acid.

In any one embodiment of the above-described embodiments, the O-acylhomoserine sulfhydrylase variant provided in the present disclosure may have substitution of the amino acid corresponding to position 4 from the N-terminus of SEQ ID NO: 1 with an amino acid selected from arginine, lysine, histidine, glutamic acid, and aspartic acid which are amino acids having electrically charged side chains. For example, the amino acid may be an amino acid selected from arginine, lysine, and histidine which are basic amino acids. In any one embodiment of the above-described embodiments, the variant may be a variant, in which the amino acid corresponding to position 4 from the N-terminus of SEQ ID NO: 1 is substituted with lysine (K).

In any one embodiment of the above-described embodiments, the O-acylhomoserine sulfhydrylase variant provided in the present disclosure may include substitution of the amino acid corresponding to position 106 from the N-terminus of SEQ ID NO: 1 with another amino acid.

In any one embodiment of the above-described embodiments, in the variant, the amino acid corresponding to position 106 from the N-terminus of SEQ ID NO: 1 may be substituted with an amino acid selected from serine, cysteine, tyrosine, asparagine, and glutamine which are polar amino acids. In any one embodiment of the above-described embodiments, the variant may be a variant, in which the amino acid corresponding to position 106 from the N-terminus of SEQ ID NO: 1 is substituted with cysteine (C).

In any one embodiment of the above-described embodiments, the O-acylhomoserine sulfhydrylase variant provided in the present disclosure may include substitution of the amino acid corresponding to position 290 from the N-terminus of SEQ ID NO: 1 with another amino acid.

In any one embodiment of the above-described embodiments, in the variant, the amino acid corresponding to position 290 from the N-terminus of SEQ ID NO: 1 may be substituted with an amino acid selected from serine, threonine, cysteine, tyrosine, asparagine, and glutamine which are polar amino acids. In any one embodiment of the above-described embodiments, the variant may be a variant, in which the amino acid corresponding to position 290 from the N-terminus of SEQ ID NO: 1 is substituted with cysteine (C).

In any one embodiment of the above-described embodiments, the O-acylhomoserine sulfhydrylase variant provided in the present disclosure may include substitution of the amino acid corresponding to position 242 from the N-terminus of SEQ ID NO: 1 with another amino acid.

In any one embodiment of the above-described embodiments, in the variant, the amino acid corresponding to position 242 from the N-terminus of SEQ ID NO: 1 may be substituted with a nonpolar amino acid. For example, the amino acid may be an amino acid selected from glycine, alanine, leucine, isoleucine, methionine, phenylalanine, tryptophan, and proline. In any one embodiment of the above-described embodiments, the variant may be a variant, in which the amino acid corresponding to position 242 from the N-terminus of SEQ ID NO: 1 is substituted with leucine (L).

In any one embodiment of the above-described embodiments, the O-acylhomoserine sulfhydrylase variant provided in the present disclosure may include substitution of the amino acid corresponding to position 261 from the N-terminus of SEQ ID NO: 1 with another amino acid.

In any one embodiment of the above-described embodiments, in the variant, the amino acid corresponding to position 261 from the N-terminus of SEQ ID NO: 1 may be substituted with a nonpolar amino acid. For example, the amino acid may be an amino acid selected from glycine, alanine, valine, isoleucine, methionine, phenylalanine, tryptophan, and proline. In any one embodiment of the above-described embodiments, the variant may be a variant, in which the amino acid corresponding to position 261 from the N-terminus of SEQ ID NO: 1 is substituted with valine (V).

In any one embodiment of the above-described embodiments, the O-acylhomoserine sulfhydrylase variant provided in the present disclosure may include substitution of the amino acid corresponding to position 395 from the N-terminus of SEQ ID NO: 1 with another amino acid.

In any one embodiment of the above-described embodiments, in the variant, the amino acid corresponding to position 395 from the N-terminus of SEQ ID NO: 1 may be substituted with a nonpolar amino acid. For example, the amino acid may be an amino acid selected from glycine, valine, leucine, isoleucine, methionine, phenylalanine, tryptophan, and proline. In any one embodiment of the above-described embodiments, the variant may be a variant, in which the amino acid corresponding to position 395 from the N-terminus of SEQ ID NO: 1 is substituted with valine (V).

In any one embodiment of the above-described embodiments, the O-acylhomoserine sulfhydrylase variant provided in the present disclosure may include one or more of substitution of the amino acid corresponding to position 4 from the N-terminus of SEQ ID NO: 1 with a basic amino acid; substitution of the amino acid corresponding to position 90 from the N-terminus of SEQ ID NO: 1 with a nonpolar amino acid; substitution of the amino acid corresponding to position 106 from the N-terminus of SEQ ID NO: 1 with a polar amino acid; substitution of the amino acid corresponding to position 242 from the N-terminus of SEQ ID NO: 1 with a nonpolar amino acid; substitution of the amino acid corresponding to position 261 from the N-terminus of SEQ ID NO: 1 with a nonpolar amino acid; substitution of the amino acid corresponding to position 290 from the N-terminus of SEQ ID NO: 1 with a polar amino acid; and substitution of the amino acid corresponding to position 395 from the N-terminus of SEQ ID NO: 1 with a nonpolar amino acid.

In any one embodiment of the above-described embodiments, the O-acylhomoserine sulfhydrylase variant provided in the present disclosure may include one or more of substitution of the amino acid corresponding to position 4 from the N-terminus of SEQ ID NO: 1 with lysine (K); substitution of the amino acid corresponding to position 90 from the N-terminus of SEQ ID NO: 1 with alanine (A); substitution of the amino acid corresponding to position 106 from the N-terminus of SEQ ID NO: 1 with cysteine (C); substitution of the amino acid corresponding to position 242 from the N-terminus of SEQ ID NO: 1 with leucine (L); substitution of the amino acid corresponding to position 261 from the N-terminus of SEQ ID NO: 1 with valine (V); substitution of the amino acid corresponding to position 290 from the N-terminus of SEQ ID NO: 1 with cysteine (C); and substitution of the amino acid corresponding to position 395 from the N-terminus of SEQ ID NO: 1 with valine (V).

In any one embodiment of the above-described embodiments, the O-acylhomoserine sulfhydrylase variant provided in the present disclosure may include one or more of substitution (A4K) of alanine (A) corresponding to position 4 from the N-terminus of SEQ ID NO: 1 with lysine (K); substitution (D90A) of aspartic acid (D) corresponding to position 90 from the N-terminus of SEQ ID NO: 1 with alanine (A); substitution (T106C) of threonine (T) corresponding to position 106 from the N-terminus of SEQ ID NO: 1 with cysteine (C); substitution of valine (V) corresponding to position 242 from the N-terminus of SEQ ID NO: 1 with leucine (L); substitution (L261V) of leucine (L) corresponding to position 261 from the N-terminus of SEQ ID NO: 1 with valine (V); substitution (H290C) of histidine (H) corresponding to position 290 from the N-terminus of SEQ ID NO: 1 with cysteine (C); and substitution of alanine (A) corresponding to position 395 from the N-terminus of SEQ ID NO: 1 with valine (V).

In any one embodiment of the above-described embodiments, the O-acylhomoserine sulfhydrylase variant provided in the present disclosure may include substitution of each of the amino acids corresponding to positions 4, 90, 106, and 290 from the N-terminus of SEQ ID NO: 1 with another amino acid.

In any one embodiment of the above-described embodiments, the O-acylhomoserine sulfhydrylase variant provided in the present disclosure may include substitution of each of the amino acids corresponding to positions 4, 90, 106, 242, 261, 290, and 395 from the N-terminus of SEQ ID NO: 1 with another amino acid.

In any one embodiment of the above-described embodiments, the O-acylhomoserine sulfhydrylase variant provided in the present disclosure may have an amino acid sequence, in which 1, 2, 3, 4, 5, 6, or 7 amino acid(s) corresponding to the positions 4, 90, 106, 242, 261, 290, and 395 from the N-terminus of SEQ ID NO: 1 is/are substituted with another amino acid(s), and which has at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, or 97% or higher homology or identity to the amino acid sequence represented by SEQ ID NO: 1.

In any one embodiment of the above-described embodiments, the O-acylhomoserine sulfhydrylase variant provided in the present disclosure may consist of an amino acid sequence of SEQ ID NO: 8, or may include an amino acid sequence having at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, or 97% or higher homology or identity thereto.

In any one embodiment of the above-described embodiments, the O-acylhomoserine sulfhydrylase variant provided in the present disclosure may consist of an amino acid sequence selected from SEQ ID NOS: 12, 13, and 14, or may have or include the amino acid sequence, or may essentially consist of the amino acid sequence. For example, the O-acylhomoserine sulfhydrylase variant may have at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% or higher, or 100% homology or identity to SEQ ID NOS: 12, 13, and 14. In the O-acylhomoserine sulfhydrylase variant, the amino acids corresponding to the positions 4, 90, 106, 242, 261, 290, and 395 from the N-terminus of SEQ ID NO: 12, 13, or 14 may be identical to those of SEQ ID NO: 12, 13, or 14.

Further, it is obvious that a variant having an amino acid sequence with deletion, modification, substitution, conservative substitution, or addition in part of the sequence may also fall within the scope of the present disclosure, as long as the variant has such homology or identity and exhibits the efficacy corresponding to that of the variant of the present disclosure.

For example, the variant may have an addition or deletion of a sequence that does not alter the function of the variant of the present disclosure at the N-terminus, the C-terminus, and/or inside the amino acid sequence, naturally-occurring mutations, silent mutations, or conservative substitutions thereof.

The “conservative substitution” refers to substitution of one amino acid with a different amino acid that has similar structural and/or chemical properties. Such amino acid substitutions may generally occur based on similarity in polarity, charge, solubility, hydrophobicity, hydrophilicity, and/or amphipathic nature of residues. Commonly, conservative substitution has little or no effect on the activity of the protein or polypeptide.

In any one embodiment of the above-described embodiments, the O-acylhomoserine sulfhydrylase variant provided in the present disclosure may further include a modification to increase the activity of O-acylhomoserine sulfhydrylase or a modification within the range of maintaining the activity.

In any one embodiment of the above-described embodiments, in the O-acylhomoserine sulfhydrylase variant provided in the present disclosure, any one or more amino acids of the amino acids corresponding to the positions 3, 65, 104, and 196 from the N-terminus of SEQ ID NO: 1 may be amino acids selected from glycine, alanine, valine, leucine, isoleucine, methionine, phenylalanine, tryptophan, proline, serine, threonine, cysteine, tyrosine, asparagine, glutamine, aspartic acid, glutamic acid, lysine, arginine, and histidine.

In any one embodiment of the above-described embodiments, in the O-acylhomoserine sulfhydrylase variant provided in the present disclosure, the amino acid corresponding to the position 3 from the N-terminus of SEQ ID NO: 1 may be isoleucine or amino acids other than isoleucine. For example, the amino acid corresponding to the position 3 may be asparagine (N).

In any one embodiment of the above-described embodiments, in the O-acylhomoserine sulfhydrylase variant provided in the present disclosure, the amino acid corresponding to the position 65 from the N-terminus of SEQ ID NO: 1 may be phenylalanine or amino acids other than phenylalanine. For example, the amino acid corresponding to the position 65 may be tyrosine (Y).

In any one embodiment of the above-described embodiments, in the O-acylhomoserine sulfhydrylase variant provided in the present disclosure, the amino acid corresponding to the position 104 from the N-terminus of SEQ ID NO: 1 may be valine (V) or amino acids other than valine. For example, the amino acid corresponding to the position 104 may be alanine (A).

In any one embodiment of the above-described embodiments, in the O-acylhomoserine sulfhydrylase variant provided in the present disclosure, the amino acid corresponding to the position 196 from the N-terminus of SEQ ID NO: 1 may be valine or amino acids other than valine. For example, the amino acid corresponding to the position 196 may be threonine (T).

In any one embodiment of the above-described embodiments, in the O-acylhomoserine sulfhydrylase variant provided in the present disclosure, the amino acids corresponding to the positions 3, 65, and 104 from the N-terminus of SEQ ID NO: 1 may be asparagine (N), tyrosine (Y), and alanine (A), respectively.

Another aspect of the present disclosure provides a polynucleotide encoding the variant of the present disclosure.

As used herein, the term “polynucleotide” refers to a DNA or RNA strand having a predetermined length or longer, as a nucleotide polymer which is a long chain of nucleotide monomers connected by a covalent bond.

For example, the polynucleotide encoding the O-acylhomoserine sulfhydrylase of the present disclosure may include the sequence encoding the polypeptide of SEQ ID NO: 1 or a sequence having 70% or more identity thereto. For example, the polynucleotide encoding the O-acylhomoserine sulfhydrylase may consist of or may essentially consist of a sequence of SEQ ID NO: 9.

In one embodiment, the polynucleotide encoding the O-acylhomoserine sulfhydrylase variant of the present disclosure may have or may include a nucleotide sequence having 70% or higher, 75% or higher, 80% or higher, 85% or higher, 90% or higher, 95% or higher, 96% or higher, 97% or higher, and less than 100% homology or identity to the sequence of SEQ ID NO: 9 or 10, may consist of or may essentially consist of a nucleotide sequence having 70% or higher, 75% or higher, 80% or higher, 85% or higher, 90% or higher, 95% or higher, 96% or higher, 97% or higher, and less than 100% homology or identity to the sequence of SEQ ID NO: 9 or 10, but is not limited thereto. In this regard, in the sequence having the homology or identity, codons encoding the amino acids corresponding to one or more positions of the positions 4, 90, 106, 242, 261, 290, and 395 from the N-terminus of SEQ ID NO: 1 may be codons encoding different amino acids from the amino acids of the positions in SEQ ID NO: 1.

Further, the polynucleotide of the present disclosure may include any sequence without limitation, as long as the sequence is able to hybridize with a probe that may be prepared from a known gene sequence, for example, a complementary sequence to the entirety or a part of the polynucleotide sequence of the present disclosure, under stringent conditions. The “stringent conditions” refers to conditions that enable specific hybridization between polynucleotides. Such conditions are specifically described in a literature (see J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York, 9.50-9.51, 11.7-11.8). For example, the conditions may include conditions under which polynucleotides having high homology or identity, such as polynucleotides having 70% or higher, 75% or higher, 80% or higher, 85% or higher, 90% or higher, 95% or higher, 96% or higher, 97% or higher, 98% or higher, or 99% or higher homology or identity hybridize with each other, but polynucleotides having lower homology or identity do not hybridize with each other; or common washing conditions for Southern hybridization, i.e., washing is conducted once, specifically twice or three times at a salt concentration and temperature corresponding to 60° C., 1×SSC, 0.1% SDS, specifically 60° C., 0.1×SSC, 0.1% SDS, and more specifically 68° C., 0.1YSSC, 0.1% SDS.

Hybridization requires that two nucleic acids have complementary sequences, although mismatches between bases may be possible depending on hybridization stringency. The term “complementary” is used to describe the relationship between nucleotide bases that can hybridize with each another. For example, with respect to DNA, adenine is complementary to thymine, and cytosine is complementary to guanine. Therefore, the polynucleotide of the present disclosure may include not only substantially similar nucleic acid sequences, but also isolated nucleic acid fragments complementary to the entire sequence.

Specifically, polynucleotides having homology or identity to the polynucleotide of the present disclosure may be detected at a Tm value of 55° C. using hybridization conditions that include a hybridization step and using the conditions described above. In addition, the Tm value may be 60° C., 63° C., or 65° C., but is not limited thereto, and may be appropriately adjusted by a person skilled in the art according to the purpose.

The appropriate stringency for hybridizing polynucleotides depends on the length of the polynucleotides and the degree of complementarity thereof, and variables thereof are well known in the art (e.g., J. Sambrook et al., supra).

Still another aspect of the present disclosure provides a vector including the polynucleotide encoding the variant of the present disclosure. The vector may be an expression vector for expressing the polynucleotide in host cells, but is not limited thereto.

The “vector” of the present disclosure may include a DNA construct that includes a nucleotide sequence of a polynucleotide encoding the target polypeptide operably linked to an appropriate expression control region (expression control sequence) to enable expression of the target polypeptide in an appropriate host cell. The expression control sequence may include a promoter capable of initiating transcription, any operator sequence for the control of such transcription, a sequence encoding an appropriate mRNA ribosome-binding domain, and a sequence controlling termination of transcription and translation. After the vector is transformed into the appropriate host cell, it may replicate or function independently of the host genome, and may be integrated into the genome itself.

The vector used in the present disclosure is not particularly limited, and any vector known in the art may be used. Examples of vectors commonly used may include a natural or recombinant plasmid, cosmid, virus, and bacteriophage. For example, pWE15, M13, MBL3, MBL4, IXII, ASHII, APII, t10, t11, Charon4A, Charon21A, etc. may be used as a phage vector or cosmid vector; and those based on pDZ, pBR, pUC, pBluescriptII, pGEM, pTZ, pCL, and pET, etc. may be used as a plasmid vector. Specifically, vectors such as pDZ, pDC, pDCM2, pACYC177, pACYC184, pCL, pECCG117, pUC19, pBR322, pMW118, pCC1BAC, pUCtk vectors, etc. may be used.

For example, a polynucleotide encoding a target polypeptide may be inserted into chromosome through a vector for intracellular chromosomal insertion. The insertion of the polynucleotide into the chromosome may be performed using any method known in the art, e.g., homologous recombination, but the method is not limited thereto. The vector may further include a selection marker for confirming its successful insertion into the chromosome. The selection marker is used for selection of cells transformed with the vector, i.e., to confirm whether the target nucleic acid molecule has been inserted, and markers which confer selectable phenotypes, e.g., drug resistance, auxotrophy, resistance to cytotoxic agents, expression of surface proteins, etc., may be used. Under the circumstances where selective agents are treated, only the cells capable of expressing the selection markers are able to survive or express other phenotypic traits, and therefore, the transformed cells can be selected.

As used herein, the term “transformation” refers to the introduction of a vector, which includes a polynucleotide encoding a target polypeptide, into a host cell or a microorganism such that the polypeptide encoded by the polynucleotide is expressed in the host cell. As long as the transformed polynucleotide can be expressed in the host cell, it may be integrated into and placed in the chromosome of the host cell, or it may be placed extrachromosomally, or irrespective thereof. Additionally, the polynucleotide includes DNA and/or RNA encoding the target polypeptide. The polynucleotide may be introduced in any form, as long as it may be introduced into the host cell and expressed therein. For example, the polynucleotide may be introduced into the host cell in the form of an expression cassette, which is a gene construct including all the elements required for its autonomous expression. In general, the expression cassette may include a promoter operably linked to the polynucleotide, transcriptional termination signals, ribosome binding sites, and translation termination signals. The expression cassette may be in the form of a self-replicable expression vector. Further, the polynucleotide may be introduced into the host cell as it is and operably linked to a sequence required for expression in the host cell, but the polynucleotide is not limited thereto.

As used herein, the term “operably linked” means that a promoter sequence, which initiates and mediates transcription of the polynucleotide encoding the variant of the present disclosure, is functionally linked to the above polynucleotide sequence.

Still another aspect of the present disclosure provides a microorganism including the variant of the present disclosure or the polynucleotide encoding the variant.

The microorganism of the present disclosure may include the variant of the present disclosure, the polynucleotide encoding the same, or the vector including the polynucleotide.

As used herein, the term “microorganism (or strain)” encompasses a wild-type microorganism or a microorganism with naturally or artificially occurring genetic modification, and may refer to a microorganism in which a particular mechanism is weakened or enhanced due to insertion of an exogenous gene or enhancement or inactivation of activity of an endogenous gene, wherein the microorganism includes genetic modification for the production of a desired polypeptide, protein, or product. The genetic modification may include enhancement and/or weakening of activity of a polypeptide involved in a particular mechanism.

The microorganism of the present disclosure may be a microorganism including any one or more of the variant of the present disclosure, the polynucleotide encoding the variant of the present disclosure, and the vector including the polynucleotide encoding the variant of the present disclosure; a microorganism which is modified to express the variant of the present disclosure or the polynucleotide encoding the variant of the present disclosure; a microorganism (e.g., recombinant strain) expressing the variant of the present disclosure or the polynucleotide encoding the variant of the present disclosure; or a microorganism (e.g., recombinant strain) having the activity of the variant of the present disclosure, but is not limited thereto.

The microorganism of the present disclosure may include any of a prokaryotic microorganism and a eukaryotic microorganism, as long as the microorganism is able to produce the O-acylhomoserine sulfhydrylase variant provided in the present disclosure, or a desired product including the O-acylhomoserine sulfhydrylase variant. For example, the microorganism of the present disclosure may include a microorganism strain of the Escherichia sp., Erwinia sp., Serratia sp., Providencia sp., Corynebacteria sp., Pseudomonas sp., Leptospira sp., Salmonellar sp., Brevibacteria sp., Hypomononas sp., Chromobacterium sp., and Norcardia sp., or fungi, or yeast. In one embodiment, the microorganism may be a microorganism strain of the Escherichia sp. or Corynebacteria sp. In any one embodiment of the above-described embodiments, the microorganism may be Escherichia coli or Corynebacterium glutamicum, but is not limited thereto.

As used herein, the term “enhancement” of activity of a polypeptide means that the activity of the polypeptide is increased, as compared to the intrinsic activity. The enhancement may be used interchangeably with terms such as activation, up-regulation, overexpression, increase, etc. In this regard, the activation, enhancement, up-regulation, overexpression, and increase may include all of those exhibiting activity that was not originally possessed or exhibiting improved activity, as compared to intrinsic activity or activity before modification. The “intrinsic activity” refers to activity of a particular polypeptide originally possessed by a parent strain or non-modified microorganism before transformation when the microorganism is transformed by genetic modification caused by a natural or artificial factor. This term may be used interchangeably with “activity before modification”. The “enhancement”, “up-regulation”, “overexpression”, or “increase” of activity of a polypeptide, as compared to intrinsic activity, means that activity and/or concentration (expression level) of a particular polypeptide is improved, as compared to those originally possessed by a parent strain or non-modified microorganism before transformation.

The enhancement may be achieved by introduction of a foreign polypeptide or enhancement of activity and/or concentration (expression level) of the intrinsic polypeptide. The enhancement of activity of the polypeptide may be identified based on the increase in the degree of activity, the expression level of the corresponding polypeptide, or the amount of a product released from the corresponding polypeptide.

Enhancement of the activity of the polypeptide may be obtained by applying various methods well known in the art, and the methods are not limited, as long as the activity of the target polypeptide is enhanced, as compared to that of the microorganism before modification. Specifically, any genetic engineering and/or protein engineering method well known in the art as routine methods of molecular biology may be used, without being limited thereto (e.g., Sitnicka et al. Functional Analysis of Genes. Advances in Cell Biology. 2010, Vol. 2. 1-16, Sambrook et al. Molecular Cloning 2012, etc.).

Specifically, the enhancement of the polypeptide of the present disclosure may be achieved by:
1) an increase in the copy number of a polynucleotide encoding the polypeptide in cells;
2) replacement of a gene expression control region on a chromosome encoding the polypeptide with a sequence having higher activity;
3) modification of a nucleotide sequence encoding an initiation codon or 5′-UTR region of a gene transcript encoding the polypeptide;
4) modification of an amino acid sequence of the polypeptide to enhance the activity of the polypeptide;
5) modification of a polynucleotide sequence encoding the polypeptide to enhance the activity of the polypeptide (e.g., modification of the polynucleotide sequence of the polypeptide gene to encode a modified polypeptide having enhanced activity of the polypeptide);
6) introduction of a foreign polypeptide exhibiting the activity of the polypeptide or a foreign polynucleotide encoding the same;
7) codon optimization of the polynucleotide encoding the polypeptide;
8) modification or chemical modification of an exposed region selected by analyzing a three-dimensional structure of the polypeptide; or
9) any combination of two or more selected from 1) to 8), without being limited thereto.

More specifically, 1) the increase in the copy number of a polynucleotide encoding the polypeptide in cells may be achieved by introduction of a vector, which may replicate and function irrespective of a host and is operably linked to a polynucleotide encoding the corresponding polypeptide, into a host cell. Alternatively, this may be achieved by introducing one copy or two or more copies of the polynucleotide encoding the corresponding polypeptide into the chromosome in a host cell. The introduction into the chromosome may be performed by introducing a vector into the host cell, the vector capable of inserting the polynucleotide into the chromosome of the host cell, but is not limited thereto. The vector is as described above.
2) The replacement of a gene expression control region (or expression control sequence) on a chromosome encoding the polypeptide with a sequence having higher activity may be, for example, mutation on the sequence by deletion, insertion, non-conservative substitution, conservative substitution, or any combination thereof to further enhance the activity of the expression control region, or replacement with a sequence having higher activity. The expression control region may include, but is not limited to, a promoter, an operator sequence, a sequence encoding a ribosome binding site, a sequence regulating termination of transcription and translation, etc., For example, the replacement may be replacement of an intrinsic promoter with a stronger promoter, but is not limited thereto.

Examples of the stronger promoter known in the art may include CJ1 to CJ7 promoters (U.S. Pat. No. 7,662,943 B2), lac promoter, trp promoter, trc promoter, tac promoter, Lambda phage PR promoter, PL promoter, tet promoter, gapA promoter, SPL7 promoter, SPL13 (sm3) promoter (U.S. Pat. No. 10,584,338 B2), O2 promoter (U.S. Pat. No. 10,273,491 B2), tkt promoter, yccA promoter, etc., but are not limited thereto.
3) The modification of a nucleotide sequence encoding an initiation codon or 5′-UTR region of a gene transcript encoding the polypeptide may be, for example, substitution with a nucleotide sequence encoding another initiation codon with a higher expression level of the polypeptide than an intrinsic initiation codon, but is not limited thereto.
4) and 5) The modification of an amino acid sequence or a polynucleotide sequence may be modification on the amino acid sequence of the polypeptide or the polynucleotide sequence encoding the polypeptide by deletion, insertion, non-conservative substitution, conservative substitution, or any combination thereof to enhance the activity of the polypeptide, or replacement with an amino acid sequence or a polynucleotide sequence which is modified to have higher activity or an amino acid sequence or a polynucleotide sequence modified to increase the activity, but is not limited thereto. The replacement may be performed by inserting the polynucleotide into the chromosome, specifically, by homologous recombination, but is not limited thereto. A vector used in the case may further include a selection marker to identify insertion into the chromosome. The selection marker is as described above.
6) The introduction of a foreign polypeptide exhibiting the activity of the polypeptide may be introduction of a foreign polynucleotide encoding the polypeptide exhibiting activity identical/similar to that of the polypeptide into a host cell. The origin and sequence of the foreign polynucleotide are not particularly limited as long as the polynucleotide exhibits activity identical/similar to that of the polypeptide. Any transformation method appropriately selected by those skilled in the art may be used for the introduction. As the introduced polynucleotide is expressed in the host cell, the polypeptide is produced, thereby increasing the activity of the polypeptide.
7) The codon optimization of the polynucleotide encoding the polypeptide may be codon optimization of an intrinsic polynucleotide to increase transcription or translation in a host cell, or codon optimization of a foreign polynucleotide for optimizing transcription and translation thereof in a host cell.
8) The modification or chemical modification of an exposed region selected by analyzing a three-dimensional structure of the polypeptide may be, for example, modification or chemical modification of an exposed region to be modified or chemically modified by comparing sequence information of a polypeptide to be analyzed with a database that stores sequence information of existing proteins, determining a template protein candidate according to similarity of the sequences, and identifying the structure based thereon.

Such enhancement of the activity of the polypeptide may be an increase in the activity or concentration/expression level of the corresponding polypeptide, as compared to the activity or concentration of a polypeptide expressed in wild-type microorganisms or microorganism strains before transformation, or an increase in the amount of a product produced from the corresponding polypeptide, without being limited thereto.

In the microorganism of the present disclosure, modification of the polynucleotide as a whole or in part may be induced by (a) homologous recombination using a vector for chromosomal insertion into the microorganism or genome editing using an engineered nuclease (e.g., CRISPR-Cas9) and/or (b) treatment with light such as UV rays and radioactive rays and/or a chemical substance, without being limited thereto. A method of modifying the gene in whole or in part may include a DNA recombination technique. For example, a part of the gene or the entire gene may be deleted by inducing homologous recombination by injecting, into the microorganism, a nucleotide sequence or vector including a nucleotide sequence having a homology with a target gene. The injected nucleotide sequence or vector may include a dominant selection marker, without being limited thereto.

As used herein, the term “weakening” of a polypeptide is a concept including both lowered activity and no activity, as compared to intrinsic activity. The weakening may be used interchangeably with terms such as inactivation, deficiency, down-regulation, decrease, reduction, attenuation, etc.

The weakening may also include the case where the activity of the polypeptide itself is reduced or eliminated, as compared to the activity of the polypeptide of the original microorganism, due to mutation of the polynucleotide encoding the polypeptide, etc., the case where the overall polypeptide activity level and/or concentration (expression level) in the cell is lower than that of the native strain due to expression inhibition of the polynucleotide encoding the same or inhibition of translation into the polypeptide, etc., the case where expression of the polynucleotide is not made at all, and/or the case where the polypeptide has no activity, even though the polynucleotide is expressed. The “intrinsic activity” refers to activity of a particular polypeptide originally possessed by a parent strain, or wild-type or non-modified microorganism before transformation, when the microorganism is transformed by genetic modification caused by a natural or artificial factor. This term may be used interchangeably with “activity before modification”. The “inactivation, deficiency, down-regulation, decrease, reduction, or attenuation” of activity of a polypeptide, as compared to intrinsic activity, means that the activity is lowered, as compared to activity of a particular polypeptide originally possessed by a parent strain or non-modified microorganism before transformation.

Specifically, such weakening of the activity of the polypeptide may be performed by any method known in the art without being limited thereto, and may be achieved by applying various methods well known in the art (e.g., Nakashima N et al., Bacterial cellular engineering by genome editing and gene silencing. Int J Mol Sci. 2014; 15 (2): 2773-2793, Sambrook et al. Molecular Cloning 2012, et al.).

Specifically, the weakening of the activity of the polypeptide of the present disclosure may be achieved by:
1) deletion of all or part of the gene encoding the polypeptide;
2) modification of the expression control region (or expression control sequence) to reduce the expression of the gene encoding the polypeptide;
3) modification of the amino acid sequence constituting the polypeptide so that the activity of the polypeptide is eliminated or weakened (e.g., elimination/replacement/addition of one or more amino acids on the amino acid sequence);
4) modification of the gene sequence encoding the polypeptide so that the activity of the polypeptide is eliminated or weakened (e.g., elimination/replacement/addition of one or more nucleotides on the nucleotide sequence of the polypeptide so as to encode the polypeptide which is modified to have no activity or weakened activity;
5) modification of a nucleotide sequence encoding an initiation codon or 5′-UTR region of a gene transcript encoding the polypeptide;
6) introduction of an antisense oligonucleotide (e.g., antisense RNA) that complementarily binds to the transcript of the gene encoding the polypeptide;
7) addition of a sequence complementary to the Shine-Dalgarno sequence to the front end of the Shine-Dalgarno sequence of the gene encoding the polypeptide so as to form a secondary structure to which ribosome binding is blocked,
8) addition of a promoter transcribed in the opposite direction to the 3′ end of the open reading frame (ORF) of the polynucleotide sequence encoding the polypeptide (Reverse transcription engineering, RTE); or
9) any combination of two or more selected from 1) to 8), without being limited thereto.

For example, 1) the deletion of all or part of the gene encoding the polypeptide may be deletion of all the polynucleotide encoding an endogenous target polypeptide in the chromosome, replacement with a polynucleotide in which some nucleotides are deleted, or replacement with a marker gene.

Further, 2) the modification of the expression control region (or expression control sequence) may be mutation on the expression control region (or expression control sequence) by deletion, insertion, non-conservative or conservative substitution, or a combination thereof, or replacement with a sequence having a weaker activity. The expression control region may include, but is not limited to, a promoter, an operator sequence, a sequence encoding a ribosome binding site, and a sequence regulating termination of transcription and translation.

Further, 5) the modification of a nucleotide sequence encoding an initiation codon or 5′-UTR region of a gene transcript encoding the polypeptide may be, for example, substitution with a nucleotide sequence encoding another initiation codon with a lower expression level of the polypeptide than an intrinsic initiation codon, but is not limited thereto.

Further, 3) and 4) the modification of an amino acid sequence or a polynucleotide sequence may be modification on the amino acid sequence of the polypeptide or the polynucleotide sequence encoding the polypeptide by deletion, insertion, non-conservative substitution, conservative substitution, or any combination thereof to weaken the activity of the polypeptide, or replacement with an amino acid sequence or a polynucleotide sequence modified to have weaker activity or an amino acid sequence or a polynucleotide sequence modified to have no activity, but is not limited thereto. For example, a stop codon is formed by introducing a mutation into the polynucleotide sequence, thereby suppressing or weakening the gene expression, but is not limited thereto.
6) The introduction of an antisense oligonucleotide (e.g., antisense RNA) that complementarily binds to the transcript of the gene encoding the polypeptide may be performed with reference to a literature [Weintraub, H. et al., Antisense-RNA as a molecular tool for genetic analysis, Reviews—Trends in Genetics, Vol. 1 (1) 1986].
7) The addition of a sequence complementary to the Shine-Dalgarno sequence to the front end of the Shine-Dalgarno sequence of the gene encoding the polypeptide so as to form a secondary structure, to which ribosome binding is blocked, may block or slow down mRNA translation.
8) The addition of a promoter transcribed in the opposite direction to the 3′ end of the open reading frame (ORF) of the polynucleotide sequence encoding the polypeptide (Reverse transcription engineering, RTE) may weaken the activity by preparing antisense nucleotides complementary to the transcript of the gene encoding the polypeptide.

Still another aspect of the present disclosure provides a method of producing an O-acylhomoserine sulfhydrylase reaction product, the method including the step of bringing O-acylhomoserine into contact with the O-acylhomoserine sulfhydrylase variant; or a microorganism including the variant or the polynucleotide encoding the same.

As used herein, the “O-acylhomoserine sulfhydrylase reaction product” refers to a substance produced by the reaction mediated by O-acylhomoserine sulfhydrylase using O-acylhomoserine as a substrate.

The O-acylhomoserine sulfhydrylase reaction product of the present disclosure includes cystathionine, homocysteine, L-methionine, acetic acid, and succinic acid.

In one embodiment, the method of producing the O-acylhomoserine sulfhydrylase reaction product may be a method of producing cystathionine, the method including the step of bringing O-acylhomoserine into contact with the O-acylhomoserine sulfhydrylase variant; or a microorganism including the variant or the polynucleotide encoding the same. The method of producing cystathionine may include the step of bringing O-acylhomoserine and L-cysteine into contact with the variant or the microorganism.

In one embodiment, the method of producing the O-acylhomoserine sulfhydrylase reaction product may be a method of producing homocysteine, the method including the step of bringing O-acylhomoserine into contact with the O-acylhomoserine sulfhydrylase variant; or a microorganism including the variant or the polynucleotide encoding the same. The method of producing homocysteine may include the step of bringing O-acylhomoserine and sulfide into contact with the variant or the microorganism.

In one embodiment, the method of producing the O-acylhomoserine sulfhydrylase reaction product may be a method of producing L-methionine, the method including the step of bringing O-acylhomoserine into contact with the O-acylhomoserine sulfhydrylase variant; or a microorganism including the variant or the polynucleotide encoding the same. For an example, the method of producing L-methionine may include the step of bringing O-acylhomoserine and methyl mercaptan into contact with the variant or the microorganism. For another example, the method of producing L-methionine may include the step of bringing O-acylhomoserine and thiosulfate into contact with the variant or the microorganism.

In any one embodiment of the above-described embodiments, the method of producing the O-acylhomoserine sulfhydrylase reaction product may be a method of producing acetic acid, the method including the step of bringing O-acylhomoserine into contact with the O-acylhomoserine sulfhydrylase variant; or a microorganism including the variant or the polynucleotide encoding the same.

In any one embodiment of the above-described embodiments, the method of producing the O-acylhomoserine sulfhydrylase reaction product may be a method of producing succinic acid, the method including the step of bringing O-succinylhomoserine into contact with the O-acylhomoserine sulfhydrylase variant; or a microorganism including the variant or the polynucleotide encoding the same.

In any one embodiment of the above-described embodiments, the O-acylhomoserine of the present disclosure may be obtained from an L-methionine precursor-producing microorganism. As used herein, the “L-methionine precursor-producing microorganism” refers to prokaryotic and eukaryotic microorganisms capable of producing an L-methionine precursor. Description of the L-methionine precursor-producing microorganism is disclosed in WO2008/013432 A1, etc. In any one embodiment of the above-described embodiments, the O-acylhomoserine may be in a purified form or in a fermentation liquid of the microorganism including O-acylhomoserine.

In any one embodiment of the above-described embodiments, the step of bringing into contact may be performed in the presence of a sulfur source. In any one embodiment of the above-described embodiments, the sulfur source may have a thiol functional group, and examples thereof may include a CH₃S— or SH— group. In any one embodiment of the above-described embodiments, the sulfur source may be selected from methyl mercaptan (CH₃SH), sodium sulfide (NaSH), thiosulfate (S₂O₃), alkanesulfonate including methanesulfonate and ethanesulfonate, sulfate, sulfite, hydrogen sulfide such as H₂S, sulfide, sulfide derivatives, organic and inorganic sulfur-containing compounds such as thioglycollate, thiocyanate, and/or thiourea, and mixtures thereof.

In any one embodiment of the above-described embodiments, the sulfur source may be a solution or gas in the form of liquid or gas. For example, when the sulfur source is methyl mercaptan, gaseous methyl mercaptan may be liquefied or dissolved in a sodium hydroxide (NaOH) solution and then used. In any one embodiment of the above-described embodiments, the methyl mercaptan may be in a liquefied sodium methyl mercaptan (CH₃S—Na) form, a gaseous or liquefied methyl mercaptan (CH₃SH) form, or a mixture of methyl mercaptan with dimethylsulfide (DMS). The mixture of DMS and methyl mercaptan is disclosed in WO2010/098629, which may be served as a reference.

In any one embodiment of the above-described embodiments, in the step of bringing into contact, a CH₃S— residue of methyl mercaptan may be substituted with a succinate residue of O-succinylhomoserine or an acetate residue of O-acetylhomoserine to produce L-methionine.

In any one embodiment of the above-described embodiments, in the step of bringing into contact, a —SH residue of sodium sulfide may be substituted with a succinate residue of O-succinylhomoserine or an acetate residue of O-acetylhomoserine to produce homocysteine.

Still another aspect of the present disclosure provides a method of producing an O-acylhomoserine sulfhydrylase reaction product, the method including the step of culturing the microorganism including the O-acylhomoserine sulfhydrylase variant; or the polynucleotide encoding the same in a medium containing O-acylhomoserine.

The O-acylhomoserine sulfhydrylase variant and the microorganism are the same as described above.

Although the medium used to culture the microorganism of the present disclosure and other culturing conditions are not particularly limited as long as the culture medium is known to be commonly used for culturing microorganisms. The microorganism of the present disclosure may be cultured in a common culture medium containing an appropriate carbon source, nitrogen source, phosphorus source, inorganic compound, amino acid, and/or vitamin under aerobic or anaerobic conditions while adjusting temperature, pH, etc.

In one embodiment, the method of producing the O-acylhomoserine sulfhydrylase reaction product of the present disclosure may include the step of recovering the O-acylhomoserine sulfhydrylase reaction product from the culture medium or the microorganism. The method of culturing the microorganism of the present disclosure may recover the desired material from the medium using an appropriate method known in the art, for example, according to a batch, continuous, or fed-batch method.

The method may further include a purification process. The purification process may be performed using an appropriate method known in the art.

### MODE FOR INVENTION

Hereinafter, the present disclosure will be described in more detail with reference to Examples and Experimental Examples. However, these Examples and Experimental Examples are for illustrative purposes only, and the scope of the present disclosure is not intended to be limited by these Examples and Experimental Examples.

### Comparative Example 1. Preparation of Rhodobacteraceae Family-Derived O-Acylhomoserine Sulfhydrylase

To prepare an O-acylhomoserine sulfhydrylase variant capable of improving methionine production, O-acylhomoserine sulfhydrylase was prepared.

A plasmid (based on pUCtk (SEQ ID NO: 11)) was prepared by obtaining nucleotide sequence information (SEQ ID NO: 10) from O-acylhomoserine sulfhydrylase (SEQ ID NO: 2) developed in WO2012/087038A2. The plasmid was transformed into E. coli K12 and cultured overnight in an LB plate medium containing 50 μg/L kanamycin, and then colonies were selected. The selected colonies were seeded in a deep 96-well plate, in which 1 mL of LB medium containing 50 μg/L of kanamycin was dispensed, and incubated overnight at 33° C. and 1,000 rpm. A part of the culture solution was mixed well with 50% glycerol to prepare a glycerol stock plate. Enzyme bacteria obtained from the prepared glycerol stock plate were seeded in a deep 96 well plate, in which 1 ml of 2×YT medium containing 50 μg/L of kanamycin was dispensed, and incubated at 33° C. and 1,000 rpm for 16 hours. The enzyme bacteria culture plate was centrifuged at 4,000G for 5 minutes using a centrifuge to remove the supernatant, and then cells were lysed using a Bugsbuster Protein Extraction Reagent (Merck) according to a method provided. The lysed cell lysate was taken and the total amount of protein was quantified using a Bio-Rad protein assay solution (BIO-Rad, USA). In addition, the expression of the protein was examined using an SDS-PAGE method. Thereafter, the recovered cell lysate was used for an enzymatic conversion reaction. The prepared protein of SEQ ID NO: 2 was used as a control in Examples below.

### Example 1: Preparation of O-Acylhomoserine Sulfhydrylase Variant-1

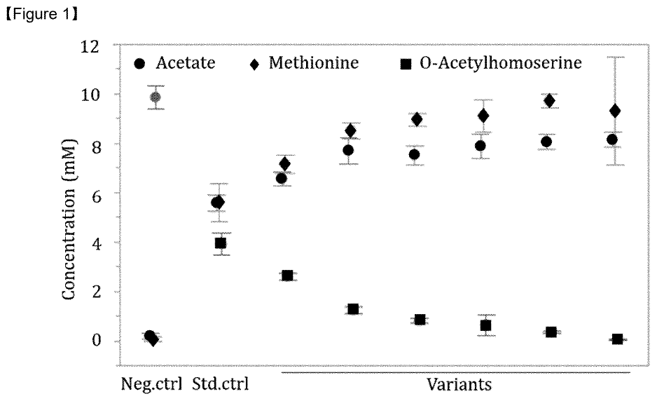

To produce an enzyme with high activity, amino acid mutagenesis was performed using a saturation mutagenesis library based on the HTP method (WO2017/100377A1). With respect to the obtained variant, a cell lysate was extracted in the same manner as in Comparative Example 1, and the activity of the variant was evaluated using methyl mercaptan (Tokyo Chemical Industry Co., Ltd., Japan) as a substrate. The experiment was performed using a sodium methyl mercaptan (CH₃S—Na, 4.7 M, 33%) solution in a liquid form, which was prepared by adding methyl mercaptan to caustic soda solution. The reaction solution for the methionine conversion reaction using sodium methyl mercaptan as a substrate is shown in Table 1, and it was carried out in a well plate with a medium depth. Immediately before the reaction, a 300 mM sodium methyl mercaptan stock solution was prepared, and 20 μL thereof was added to 180 μL of the reaction solution, followed by mixing well. 6 μL of the enzyme extract was added, and left in an incubator at 40° C. for 1 hour, transferred to a TCA plate, and the final reaction was completed. The completed reaction solution was evaluated for the conversion activity of the enzyme by measuring concentrations of methionine, acetic acid, and O-acetylhomoserine (OAH) through an HPLC method. Activity is shown in FIG. 1 by comparing with those of a control (standard control) containing the enzyme prepared in Comparative Example 1 and a negative control not containing O-acylhomoserine sulfhydrylase.

TABLE 1

| | Component | Volume (mL) |
|---|---|---|
| Citrate-Phosphate | 0.2M Sodium phosphate | 167.4 |
| buffer (pH 5.0) | dibasic (Na₂HPO₄) | |
| | 0.1M Citric acid | 157.6 |
| Substrate | 50 g/L OAHS | 12.2 |
| Coenzyme | 1 mM PLP | 3.4 |

A strain containing the variant with the highest enzyme activity was selected, a plasmid was obtained therefrom, and a nucleotide sequence thereof was analyzed through a sequencing technique. As a result of the nucleotide sequence analysis, the variant was identified as a variant (SEQ ID NO: 12), in which an amino acid residue aspartic acid (D) at position 90 in the O-acylhomoserine sulfhydrylase of SEQ ID NO: 2 was substituted with alanine (A). This variant was named D90A variant.

### Example 2: Preparation of O-Acylhomoserine Sulfhydrylase Variant-2

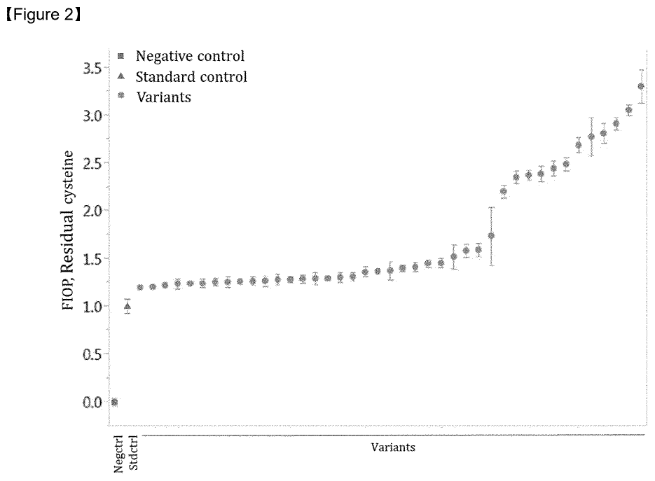

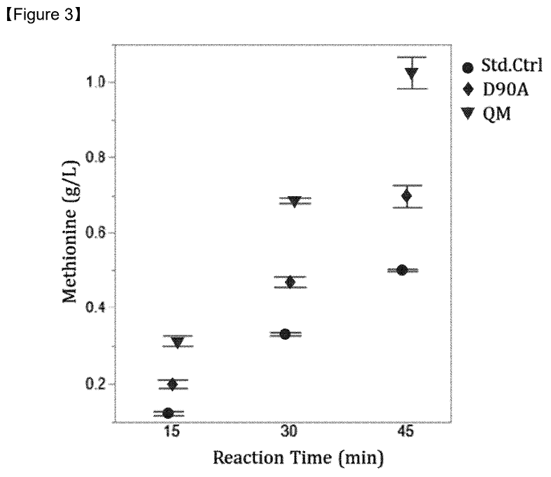

Based on the saturation mutagenesis library prepared in Example 1, a combinatorial variant was prepared (WO2017100377A1) using the combinatorial consolidation mutation of the variants showing excellent activity. The conversion activity was evaluated for the prepared variants in the same manner as in Example 1, and the activities of the variants are shown in FIG. 2. Among them, the most effective variant was selected and named QM variant. The methionine conversion experiment using methyl mercaptan as a substrate as in Example 1 was performed for the QM variant. As a result, it was confirmed that the methionine conversion ability of the QM variant was further improved, as compared to the control or the D90A variant of Example 1. As shown in FIG. 3, it was confirmed that when the QM variant was used, the production of methionine was highest over time. As a result of analyzing the amino acid sequence of the QM variant, the QM variant was confirmed to include A4K, D90A, T106C, and H290C mutations (SEQ ID NO: 13), as compared to the control.

### Example 3: Evaluation of Stability of Variant Under Different pH Conditions

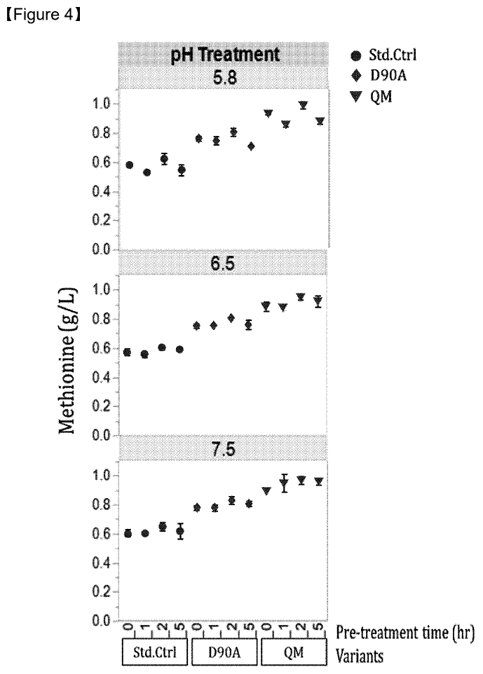

To examine the enzyme stability of the selected variants, D90A and QM, under each pH condition, the cell lysate obtained in the same manner as in Comparative Example 1 was exposed to reaction solutions at pH 5.8, 6.5, and 7.5, respectively, for 0, 1, 2, and 5 hours. Then, the methionine conversion experiment using methyl mercaptan as a substrate as in Example 1 was performed. As a result, it was confirmed that the QM variant showed the highest activity as a result of the enzyme reaction exposed to pH 5.8, 6.5, and 7.5 conditions, as shown in FIG. 4. In addition, the enzyme activity was maintained regardless of the exposure time of the cell lysate. This indicates that the variant discovered in the present disclosure stably maintains the high activity under various pH conditions.

### Example 4: Preparation of O-Acylhomoserine Sulfhydrylase Variant-3

Based on the QM variant selected in Example 2, secondary combinatorial variants were obtained through the combinatorial consolidation mutation of the variants showing excellent activity in Example 1 in the same manner as in Example 2.

With respect to the obtained variants, a methionine conversion experiment using acetyl homoserine and sodium methyl mercaptan as substrates was performed under each pH condition. Among the variants, the most excellent variant was named Variant 3.

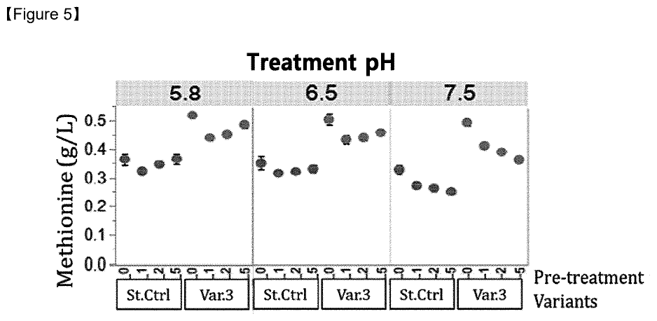

As shown in FIG. 5, Variant 3 showed the highest conversion activity and stability of the enzyme activity under all pH conditions, as compared to the control. As a result of analyzing the amino acid sequence of Variant 3, Variant 3 was confirmed to include A4K, D90A, T106C, V242L, L261V, H290C, and A395V mutations (SEQ ID NO: 14), as compared to the control of Comparative Example 1.

### Example 5: Evaluation of Scale-Up Activity of Methionine Conversion Reaction of Selected Variant

Flask and 30-liter reactor system tests were performed for the purpose of evaluating superiority of the activity of O-acylhomoserine sulfhydrylase Variant 3 selected in Example 4 in a scale-up system.

### 5-1. Production of Enzyme Extract for Flask Test

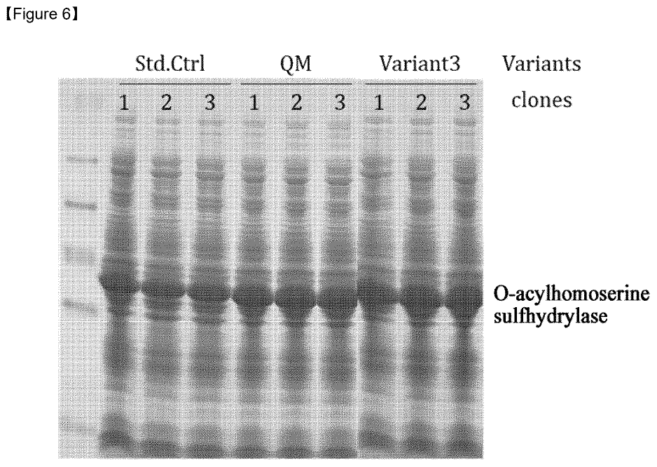

To prepare enzyme extracts of control, QM variant, and Variant 3, E. coli K12 cells, each containing a gene vector encoding each of the enzymes, were plated on an LB plate medium containing 50 μg/L kanamycin, and incubated in an incubator at 30° C. overnight. All strains of the plate medium cultured overnight were collected and seeded in 50 mL of 2×YT medium containing 1% glucose containing 50 μg/L of kanamycin, and then incubated at 33° C., 200 rpm for 16 hours. 5 mL of the enzyme culture was measured for OD₆₀₀ value, and a part thereof was taken for identifying the protein sample through SDS-PAGE, and the remaining enzyme culture was centrifuged at 4000G for 5 minutes using a centrifuge to remove the supernatant. The remaining pellet was suspended in 5 mL of 100 mM PBS (pH 7.5), and 500 μL thereof was aliquoted in each well of a 96 deep well plate, followed by treatment with 10 μl of xylene, and stored in a shaking incubator at 1150 rpm for 60 minutes. 60 μL of the xylene-treated sample was taken and mixed with 540 L of 100 mM PBS (pH 7.5) to prepare a diluent of enzyme disruption to be used in the enzyme conversion reaction, and the total amount of protein was quantified using a Bio-Rad protein assay solution (BIO-Rad, USA). In addition, the protein expression was examined using the SDS-PAGE method. As a result of protein expression of each enzyme variant clone, it was confirmed that the protein expression levels of the variant QM and variant 3 were superior to that of the control under the same OD₆₀₀ condition, as shown in FIG. 6.

### 5-2. Evaluation of Conversion Activity of Flask-Cultured Enzyme

The conversion titer was measured using the cell lysate obtained by the method of Example 5-1 and a sodium methyl mercaptan solution. A citric acid-phosphate buffer consisting of 0.1 M citric acid and 0.2 M sodium phosphate dibasic (Na₂HPO₄) was prepared according to pH 7.5, pH 6.5, and pH 5.7, and the conversion reaction solution was prepared according to the composition shown in Table 2. At this time, the conversion reaction solution, except for the sodium methyl mercaptan solution, was first mixed and incubated at 40° C. for 5 minutes, and then the reaction was started while adding sodium methyl mercaptan thereto.

TABLE 2

| | Volume (μL) |
|---|---|
| Citrate-Phosphate buffer (each pH) | 153 |
| 1.5M OAHS | 89.15 |
| 10 mM PLP | 3.75 |
| Enzyme | 1.25 |
| 21% SMM (Sodium methylmercaptan) | 2.85 |
| Total reaction solution | 250 |

1 hour after the start of the reaction, the reaction was terminated, and OAH and methionine concentrations were measured using HPLC to determine the enzyme conversion activity, as shown in Table 3. The number of enzyme units was calculated by mg/mL/min as the conversion rate per minute of methionine.

TABLE 3

| | Specific Activity (units/mL) | | |
|---|---|---|---|
| Variants | pH 7.5 | pH 6.5 | pH 5.7 |
| Std. Ctrl | 381.4 | 96.8 | 23.6 |
| QM | 711.4 | 208.3 | 64.0 |
| Variant 3 | 862.6 | 273.5 | 84.7 |

As a result, it was confirmed that Variant 3 showed the most excellent enzyme activity under each pH condition.

### 5-3. Production of Enzyme Extract in 30 Liter Fermenter and Conversion Reaction Method

This Example was performed using a 30 L batch reactor (CNS Co., Ltd., 30 liter liquid fermenter) for the purpose of evaluating the conversion activity of the O-acylhomoserine sulfhydrylase variant identified in the flask by scale-up of the reactor system for the methionine enzymatic conversion. For the production and conversion reaction of the enzyme extract, a method of producing L-methionine from an O-acetylhomoserine culture solution, specified in the existing patent (WO2012/087038A2), was employed.

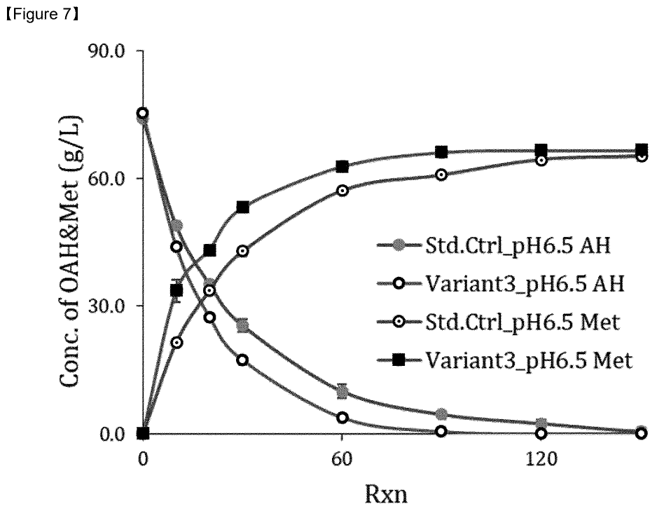

The graph of methionine conversion by the reaction is shown in FIG. 7, indicating that the conversion activity of Variant 3 was higher than that of the existing O-acylhomoserine sulfhydrylase even under the scale-up condition.

As a result of evaluating the conversion activity under pH 5.7 condition in the same manner as described above, it was confirmed that the conversion activity of the control was 87%, whereas the conversion rate of Variant 3 was 100%, and the reaction time thereof was further shortened.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

1. An O-acylhomoserine sulfhydrylase variant, wherein one or more of amino acids corresponding to positions 4, 90, 106, 242, 261, 290, and 395 from the N-terminus of SEQ ID NO: 1 are each substituted with another amino acid.

2. The O-acylhomoserine sulfhydrylase variant of claim 1, wherein the O-acylhomoserine sulfhydrylase is acylhomoserine sulfhydrylase derived from a microorganism of the family Rhodobacteraceae.

3. The O-acylhomoserine sulfhydrylase variant of claim 1, wherein amino acids corresponding to positions 3, 65, and 104 from the N-terminus of SEQ ID NO: 1 are each substituted with another amino acid.

4. The O-acylhomoserine sulfhydrylase variant of claim 1, wherein amino acids corresponding to positions 4, 90, 106, and 290 from the N-terminus of SEQ ID NO: 1 are each substituted with another amino acid.

5. The O-acylhomoserine sulfhydrylase variant of claim 4, wherein amino acids corresponding to positions 242, 261, and 395 from the N-terminus of SEQ ID NO: 1 are each substituted with another amino acid.

6. The O-acylhomoserine sulfhydrylase variant of claim 1, wherein the variant includes one or more of substitution of the amino acid corresponding to position 4 from the N-terminus of SEQ ID NO: 1 with a basic amino acid; substitution of the amino acid corresponding to position 90 from the N-terminus of SEQ ID NO: 1 with a nonpolar amino acid; substitution of the amino acid corresponding to position 106 from the N-terminus of SEQ ID NO: 1 with a polar amino acid; substitution of the amino acid corresponding to position 242 from the N-terminus of SEQ ID NO: 1 with a nonpolar amino acid; substitution of the amino acid corresponding to position 261 from the N-terminus of SEQ ID NO: 1 with a nonpolar amino acid; substitution of the amino acid corresponding to position 290 from the N-terminus of SEQ ID NO: 1 with a polar amino acid; and substitution of the amino acid corresponding to position 395 from the N-terminus of SEQ ID NO: 1 with a nonpolar amino acid.

7. The O-acylhomoserine sulfhydrylase variant of claim 1, wherein the variant includes one or more of substitution of the amino acid corresponding to position 4 from the N-terminus of SEQ ID NO: 1 with lysine (K); substitution of the amino acid corresponding to position 90 from the N-terminus of SEQ ID NO: 1 with alanine (A); substitution of the amino acid corresponding to position 106 from the N-terminus of SEQ ID NO: 1 with cysteine (C); substitution of the amino acid corresponding to position 242 from the N-terminus of SEQ ID NO: 1 with leucine (L); substitution of the amino acid corresponding to position 261 from the N-terminus of SEQ ID NO: 1 with valine (V); substitution of the amino acid corresponding to position 290 from the N-terminus of SEQ ID NO: 1 with cysteine (C); and substitution of the amino acid corresponding to position 395 from the N-terminus of SEQ ID NO: 1 with valine (V).

8. A polynucleotide encoding the variant of claim 1.

9. A microorganism comprising an O-acylhomoserine sulfhydrylase variant of claim 1, or a polynucleotide encoding the variant.

10. A method of producing an O-acylhomoserine sulfhydrylase reaction product, the method comprising the step of bringing O-acylhomoserine into contact with an O-acylhomoserine sulfhydrylase variant of claim 1 or a microorganism including the variant or a polynucleotide encoding the same.

11. The method of claim 10, wherein the O-acylhomoserine sulfhydrylase reaction product is selected from L-methionine, acetic acid, succinic acid, homocysteine, and cystathionine.

12. A method of producing an O-acylhomoserine sulfhydrylase reaction product, the method comprising the step of culturing, in a medium containing O-acylhomoserine, a microorganism including the O-acylhomoserine sulfhydrylase variant of claim 1; or a polynucleotide encoding the same.

13. The O-acylhomoserine sulfhydrylase variant of claim 1, wherein the variant comprises an amino acid sequence having 90% or more sequence identity to the polypeptide of SEQ ID NO: 1.
